(19) United States
(12) Patent Application Publication
(10) Pub. No.: US 2019/0191796 A1
(43) Pub. Date: Jun. 27, 2019

(54) Composition and Methods for Enhancing the Application of Materials on an Elastomeric article and Article Thereof

(71) Applicant: SHEN WEI (USA) INC., Union City, CA (US)

(72) Inventors: Belle L. Chou, Union City, CA (US); Sisitha Sudarshana, Athurugiriya (LK); Amila Suranga, Athurugiriya (LK)

(21) Appl. No.: 15/855,977

(22) Filed: Dec. 27, 2017

(51) Int. Cl. A41D 19/00; A61K 8/25; A61K 8/81; A61K 8/02; A61K 8/92; A61Q 15/00; A61M 35/00; A61Q 19/00; A61K 8/73; A61K 8/26

(52) U.S. Cl. CPC A41D 19/0082; A61K 8/25; A61K 8/8147; A61K 8/0233; A61K 8/0208; A61K 8/92; A61K 2800/87; A61M 35/00; A61Q 19/007; A61K 8/731; A61K 8/26; A61K 2800/805; A61Q 15/00

(57) ABSTRACT

Disclosed are compositions and methods of enhancing the application of subsequent treatment on an elastomeric skin protection article and articles thereof. Embodiments disclose processes, including creating microscale voids and cracks, adding a surface activator, and rearranging or altering the inside surface of a glove. Subsequent treatment of a material, such as soothing agents, pH controlling substances, herbal oils and powders can coat the inside surface of a glove in a smaller quantity and last longer when added after the glove's inside surface has undergone the enhancing method.

### BACKGROUND OF THE INVENTION

### 1. Field of the Invention

The exemplary embodiment(s) of the present invention generally relates to an elastomeric skin protection article. More specifically, the exemplary embodiment(s) of the present invention relates to methods of enhancing the application of subsequent treatment on a hand protection article.

### 2. Background

Hand protection articles such as gloves are designed for protecting the user from contaminations, physical hazards and various exposures while used in a wide range of activities. Sweaty and hot hands, allergic reactions, unusual dry skin are common problems faced by frequent glove users. These negative side effects create an unpleasant user experience and discourage users to wear gloves. Subsequent material such as herbal treatments, soothing materials, and medication have been introduced to reduce these negative effects and create a better user experience; however, one challenge is creating a subsequent material compatible with the polymeric backbone of the glove so that excess material is not wasted to coat the glove. As a result, subsequent materials tend not to be as effective and manufacturers lose money from overloading formulations on the glove.

An example for adding subsequent material to a glove is applying a dry powdered aloe material to the inside surface of a glove. However, there ends up being visual powder residue along with the risk of inhaling the powder while using the gloves. There are also concerns of delamination of the layers, increase in cost of manufacturing, and increased thickness/bulkiness of the gloves. There is an existing process of manufacturing a glove by coating the inside surface with an aqueous solution of aloe-vera and then drying it to remove the aqueous solution. However, this process can encourage microbial growth if not adequately dried.

Accordingly, there is a need to minimize the incompatibility of the inside surface of a hand protection article, such as gloves, with a subsequent treatment in order to create a pleasant user experience.

### SUMMARY

According to an embodiment of the present invention, there is an elastomeric article comprising at least one outer layer and at least one inside layer, the inside layer having a composition that enhances an application of a subsequent treatment/material to a dried skin-facing surface of the inside layer for increased effectiveness of the subsequent treatment/material on the skin.

According to an embodiment of the present invention, the inside layer has a composition comprising a plurality of surface voids and cracks developed by at least one substance that is evaporated to create the surface voids and cracks at a temperature where polymer cross links are generated. In an embodiment, the at least one substance is a semi-reversible water absorption material.

According to an embodiment of the present invention, the inside layer has a composition comprising one or more substances bonded with a delayed surface activator, hereinafter “bonded mixture”, the bonded mixture migrated to the skin-facing surface of the inside layer, and bondable with the subsequent material.

In further embodiments of the present invention, the delayed surface activator is a silane coupling agent. In another embodiment, the one or more substances include a surface migration agent.

According to another embodiment of the present invention, the inside layer has a composition that enhances an application of a subsequent film to a dried skin-facing surface of the inside layer for increased effectiveness of the subsequent film on the skin. The composition comprises a surface preparation mixture including a coalescing agent, the mixture generating a swollen surface upon which the coalescing agent is distributed, increasing adherability of the subsequent film to the swollen surface. In a further embodiment, the surface preparation mixture is a blend of organic acids, inorganic acids, and organic solvents.

These features, advantages and other embodiments of the present invention are further made apparent, in the remainder of the present document, to those of ordinary skill in the art.

### BRIEF DESCRIPTION OF THE DRAWINGS

In order to more fully describe embodiments of the present invention, reference is made to the accompanying drawings. These drawings are not to be considered limitations in the scope of the invention, but are merely illustrative.

FIG. 1 illustrates a process for creating surface voids and cracks, according to an embodiment of the present invention.

FIG. 2 illustrates micro level void on the inside surface of a glove after removal of absorbed water molecules, according to an embodiment of the present invention.

FIG. 3 illustrates another micro level void on the inside surface of a glove after removal of absorbed water molecules, according to an embodiment of the present invention

FIG. 4 illustrates selective crack patterns created by evaporating absorbed water, according to an embodiment of the present invention.

FIG. 5 illustrates efficiency of material absorbance to the glove surface with micro level voids, according to an embodiment of the present invention.

FIG. 6 illustrates a process for adding a delayed surface activator, according to an embodiment of the present invention.

FIG. 7 illustrates a silane bond between a rubber matrix and delayed surface activator material, according to an embodiment of the present invention.

FIG. 8 is a graph illustrating the effective loading of subsequent material and time, according to an embodiment of the present invention.

FIG. 9 is a graph illustrating the effectiveness of a delayed surface activator, according to an embodiment of the present invention.

FIG. 10 illustrates a process for surface alteration of a composite, according to an embodiment of the present invention.

FIG. 11 is a graph illustrating the efficiency of material absorbance using surface altering chemical composite, according to an embodiment of the present invention.

### DETAILED DESCRIPTION OF SPECIFIC EMBODIMENTS

The description above and below and the drawings of the present document focus on one or more currently preferred embodiments of the present invention and also describe some exemplary optional features and/or alternative embodiments of the present invention. The description and drawings are for the purpose of illustration and not limitation. Those of ordinary skill in the art would recognize variations, modifications, and alternatives. Such variations, modifications, and alternatives are also within the scope of the present invention. Section titles are terse and are for convenience only.

Various embodiments of the present invention illustrated in the drawings may not be drawn to scale. Rather, the dimensions of the various features may be expanded or reduced for clarity. In addition, some of the drawings may be simplified for clarity. Thus, the drawings may not depict all of the components of a given apparatus (e.g., device) or method. Unless the meaning is clearly to the contrary, all ranges set forth herein are deemed to be inclusive of the endpoints.

When the glove is stripped off from the former the outside surface of the glove becomes the inside, skin-facing surface of the finish glove product. The outside surface can be referred to as the outer surface, outside layer, or the outer layer. The inside surface can be referred to as the inside layer or the inner surface which faces the wearer's skin. Other polymeric materials such as natural rubber, butyl, and vinyls can be used also. Subsequent material can also be referred to as secondary material. The use of the term secondary material does not limit the order in which the material needs to be applied. Microscale waves, hollow voids, cavities and cracks refer to increased surface area and volume.

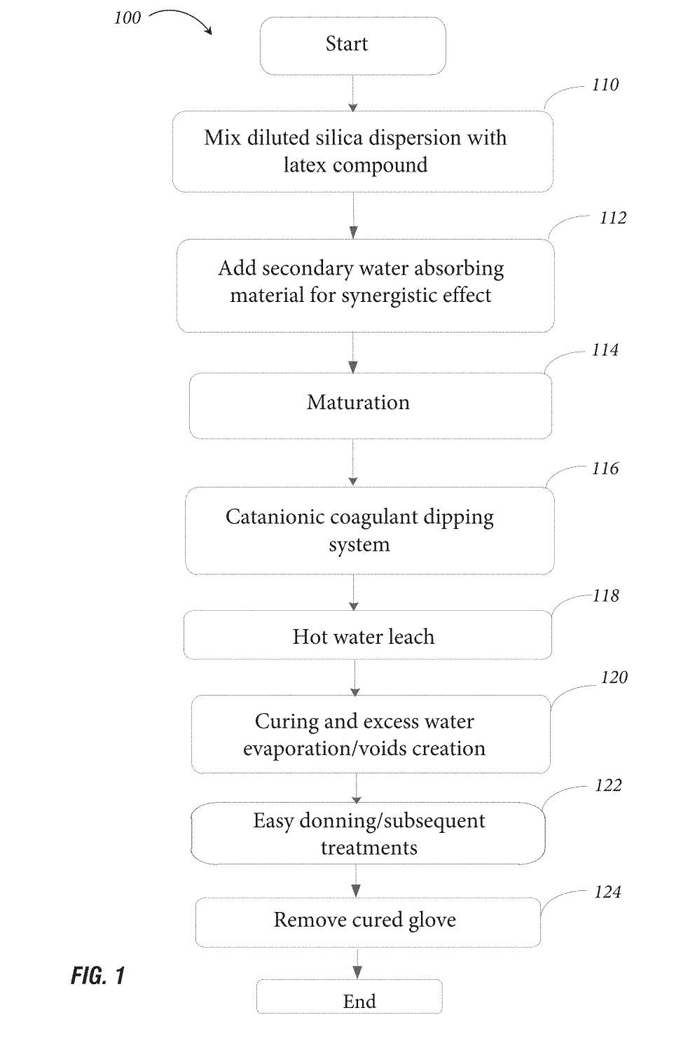

Referring now to FIG. 1, which illustrates a process 100 for creating surface hollow voids, microscale waves, and cracks, according to an embodiment of the present invention. The creation of selective cavities on the inside surfaces of the glove increases the effectiveness of a subsequent treatment in terms of the quantity applied by to the glove. The effectiveness is increased because less subsequent treatment is applied to the inside surface of the glove. The hollow voids are created by incorporating a material which undergo water absorption and exudation which is a reversible reaction under thermal treatment. The absorbed water and saturated material are incorporated into the inside surface and absorbed water is exuded during the curing process generating micro level voids, cracks and microscale waves at the inside surface of the glove. This process increases the surface area and volume of the inside surface by creating selective cavities by introducing one or more substances which can emit one or more gaseous substances, evaporate or sublimate creating surface voids and cracks at the temperature where polymer cross links are generated. As a result, a smaller amount of the subsequent treatment is needed to be effective. Subsequent treatment is then applied on both the inside surface and in between the micro level cracks and voids. Subsequent treatment with a higher dosage can be deposited on the micro level cracks and voids at a smaller quantity than deposited on a conventional glove. The micro level cracks and voids prevent the subsequent treatment from rubbing off when donning or doffing the gloves, therefore the effects of the subsequent material is long lasting compared to conventional gloves.

In step 110, diluted silica dispersion is mixed with a latex compound. In step 112, subsequent water absorbing material is added to the mixture in step 110 for a synergistic effect. Step 112 is optional. In step 114, maturation occurs. In step 116, a catanionic coagulant dipping system is used. In step 118, hot water leach occurs. In step 120, curing occurs and excess water is evaporated and voids are created. In step 122, easy donning/post treatments of the glove occurs. In step 124, the cured glove is removed from the former.

As is appreciated by those skilled in the art, semi-reversible water absorption materials have higher affinity to water and usually have higher water absorbing capacity compared to its original weight. One advantage of using semi-reversible water absorption material is its ability to release some absorbed water in certain conditions because the material is partially reversible. An example of a semi-reversible water absorption material is fumed silica. It is appreciated by those skilled in the art that silica has an extraordinary high surface area. Hydrophilic fumed silica particles can absorb considerable amounts of water. This water absorption is partially or fully reversible.

Step 110 is an aqueous dispersion of fumed silica prepared using high speed stirring with a dispersing agent. The aqueous dispersion in step 110 can be made at about 2%-10% w/w, preferably 5%-7% w/w. The aqueous dispersion is prepared at forced conditions because it is not fully soluble under general conditions. At forced conditions the mixing temperature can be maintained between 40-80 degrees Celsius, preferably between 60-80 degrees Celsius and the stirring speed can be maintained within 3000-10000 revolutions per minute (“rpm”) and the mixing can be continued for preferably 90-120 minutes. Stirring occurs while the aqueous dispersion of fumed silica is slowly and continuously added to the initial latex compound. Once the aqueous dispersion is fully added, the mixture is stirred for about 1-2 hours at a constant speed.

In optional step 112, additional types of water absorbing material in the form of dilute aqueous solution can be slowly added under continuous stirring conditions to the mixture in step 110 to create a synergistic effect in altering the surface of the base polymer. Powdered acid soluble materials such as inorganic carbonates can be used instead of water absorbing materials. An example for a synergistic effect observed by adding two water absorbing materials is the addition of poly acrylate salt solution together with methyl cellulose solution. This can provide better outcome for creating microscale voids and cracks. These powdered acid soluble materials can be selectively washed off by any suitable solution in order to create voids. The presence of an aqueous dispersion of fumed silica at about 5% w/w, with methyl cellulose solution at about 0.5%-1% w/w, provides a better outcome compared to having them individually in the formulation in creating the desired surface alteration. Alternatively, the synergistic effect could be obtained by other mixtures of fumed silica selected from: poly vinyl alcohol, hydrated poly vinyl acetate, sodium poly acrylate, carboxylated methyl cellulose, polyacrylamide, polyacrylate, polyacrylic acid, polymethacrylate. Other alternative mixtures of fumed silica can be fumed silica dispersion and poly vinyl acetate at about 8:3 w/w %. Yet another alternative mixture of fumed silica can be fumed silica dispersion and sodium poly acrylate at about 5:1 w/w %. Yet another further alternative mixture of fumed silica can be fumed silica, carboxy methyl cellulose with poly vinyl acetate at about 6:3:1 w/w %.

In step 114, a 24-48-hour maturation step occurs at room temperature, which is approximately at 20-40 Celsius. The preparation of the polymer solution is completed by adding anti-webbing agents, curing agents and pigments. Anti-webbing agents prevent thin web formation between fingers. Pigments give color to the article and curing agents such as sulphur, accelerators and activators are used. Prepared polymeric solution can be kept for desired maturation in order to create intra molecular cross links.

In step 116, the matured polymeric solution in step 114 can be used in preparing gloves. The catanionic coagulant is a mixture of divalent cation salt, wetting agent, and parting agent. The solvent can be water or alcohol. The divalent cation salt can be calcium chloride, or calcium nitrate. While on the former, the outside surface of the glove will later become the inside surface of a glove when the glove is taken off the former. As such, the outside surface while on the former comprises a continuous polymeric compound. In step 116, the outside surface of the glove 2 while on the former can be dipped into the matured polymeric solution of step 114.

Hot water leaching occurs in step 118. In this step, dipped gloves on the former are placed in warm water for a couple of minutes to remove water soluble materials such as excess stabilizers and coagulants.

In step 120, a continuous gelled surface is fixed from the coagulant and it starts to dry inside the oven. The outside surface can comprise a mixture of water absorbed materials, methyl cellulose, and poly acrylate salt. With the humidity difference and oven temperature, bonded water molecules detach from the polymeric matrix and start to evaporate. The drying of the wet gelled surface starts create a relatively sealed outer surface compared to the inside surface attached to the former. When the trapped water on the outside surface starts to evaporate causing increase pressure, micro level voids and cracks are created on the outside surface thus increasing the surface area and volume of the outside surface.

In step 122, subsequent treatment can occur after the dried glove 2 on the former comes out of the oven. Subsequent treatment can be applied to the glove 2 while they are on the production line/on the former or after the gloves are stripped from the former/off the production line. The subsequent treatment helps with the donning of the finished glove. The subsequent treatment 122 can be an application of a polymer, powder, or solution as desired, for example, oil and waxes, natural herbal extracts, therapeutic agents, soothing agent, a diluted acid wash, chlorination, polymer coating, cleansing agent, synthetic or natural flock and antiperspirant materials. Small micro-level cracks and voids create additional space and volume and the particles of the subsequent treatment can occupy this additional space. The micro-level cracks and voids hinder easy removal of subsequent treatment due to the enhance space between the cracks and voids. In step 124, after leaving the oven, the cured glove 2 can be stripped off the former and the outside surface with the micro voids and cracks and subsequent treatment will become the inside surface of the glove.

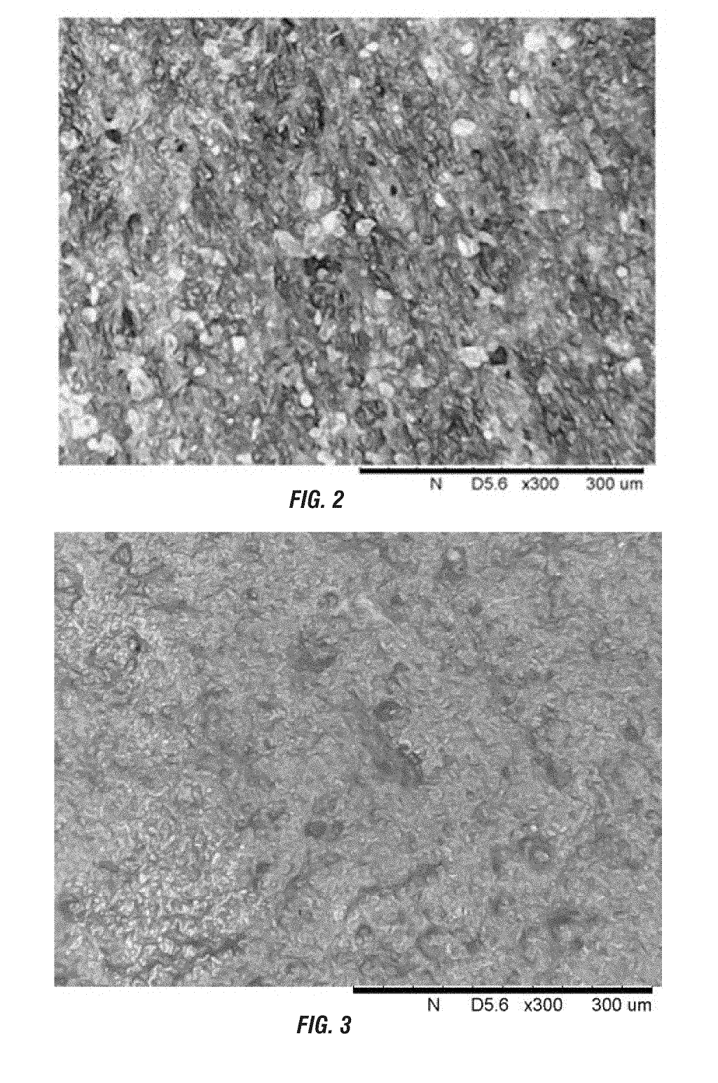

FIGS. 2-4 illustrate several images of the inside surface of the glove after undergoing the method described in FIG. 1. FIGS. 2 and 3 illustrate micro-level void on the inside surface of a glove after removal of absorbed water molecules, according to an embodiment of the present invention. The inside glove surfaces contain a compound of aqueous methyl cellulose and dilute fumed silica in 5:1 weight ratio. The total material percentage is about 0.5%-2.0%. The solution maturation time is about 24 hours in FIG. 2 and about 36 hours in FIG. 3.

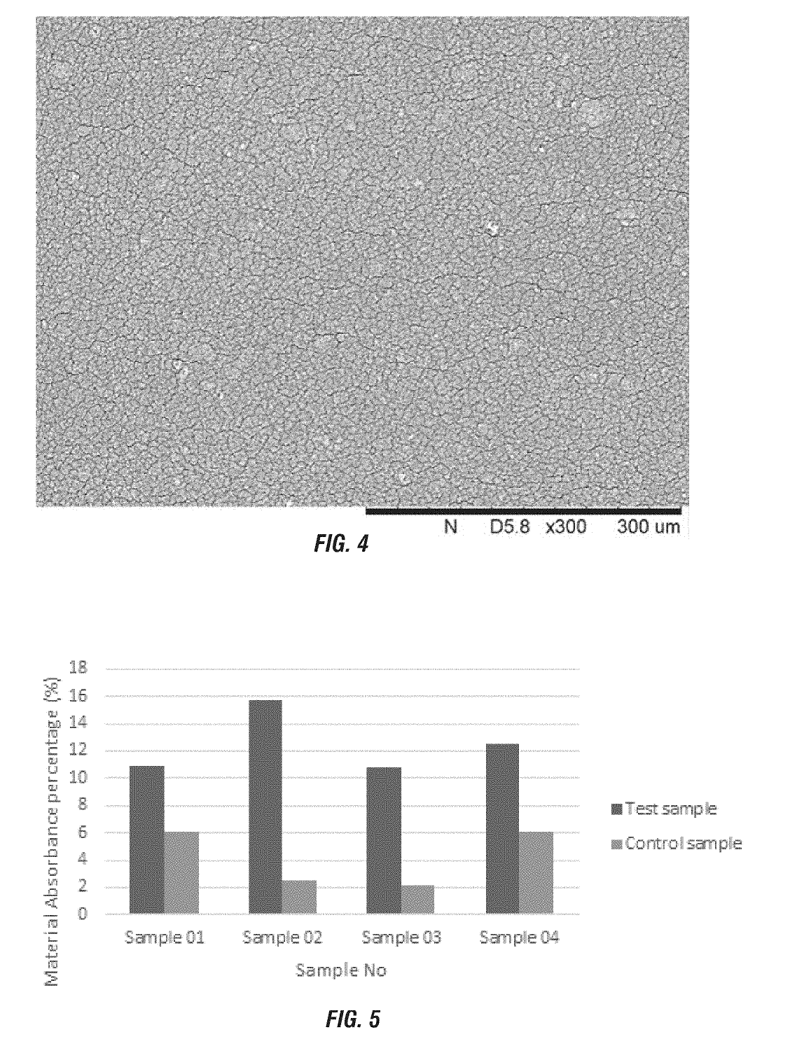

FIG. 4 illustrates selective crack patterns created by evaporating absorbed water, according to an embodiment of the present invention. The compound on the inside surface of the glove contains precipitated calcium carbonate. The coating illustrated in FIG. 4 was treated with 0.5% diluted hydrochloric acid before the subsequent treatment.

FIG. 5 illustrates secondary treatment efficiency of material absorbance to the glove surface with micro level voids, according to an embodiment of the present invention. An experiment was conducted to evaluate the effectiveness of the voids created by evaporating absorbed water. The inside surface of the test sample was created according to the above-mentioned procedure containing micro level voids. (See Table 1). The control sample is a regular glove with similar dimensions. (See Table 2). Prepared glove pieces having micro level voids were soaked in aqueous soothing agent under similar conditions. Both soaked glove pieces were dried to remove excess moisture. Weight gain was measured on those dried glove pieces.

TABLE 1
Test sample

| W1 | W2 | W2 − W1 | (W2 − W1)/W1 % |
|---|---|---|---|
| 0.3709 | 0.4115 | 0.0406 | 10.946 |
| 0.3728 | 0.4314 | 0.0586 | 15.719 |
| 0.3919 | 0.434 | 0.0421 | 10.743 |
| 0.3999 | 0.4501 | 0.0502 | 12.553 |

W1—weight of the glove film
W2—Glove film + soothing material

TABLE 2
Control

| W1 | W2 | W2 − W1 | (W2 − W1)/W1 % |
|---|---|---|---|
| 0.4848 | 0.514 | 0.0292 | 6.02310231 |
| 0.4771 | 0.489 | 0.0119 | 2.494236009 |
| 0.4089 | 0.4176 | 0.0087 | 2.127659574 |
| 0.4423 | 0.4691 | 0.0268 | 6.059235813 |

W1—weight of the glove film
W2—Glove film + soothing material

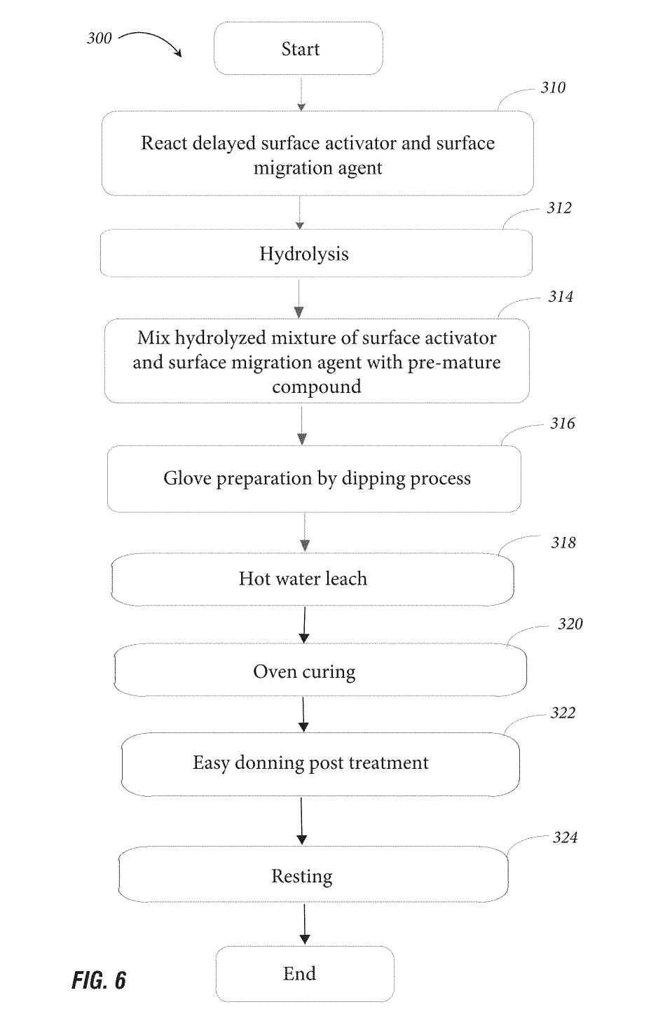

Referring now to FIG. 6, which illustrates a process 300 for adding a delayed surface activator, according to an embodiment of the present invention. This process is another embodiment of minimizing the incompatibility of the inside surface with a subsequent treatment. This process uses a substance that migrates to the inside surface while bonded to a delayed surface activator which hereinafter may be referred to as the “bonded mixture”. The delayed surface activator is a chemical substance that has better affinity to the subsequent material. Examples of subsequent material can be soothing agents, pH controlling substances, herbal oils and powders are added to the inside surface of a glove to enhance ease of donning, hand health, and reduce sweating. In most circumstances, these materials do not have an effective affinity to the polymeric layer of a glove. As a result, they are loosely coated and do not perform any long-lasting effect during use.

An example of a delayed surface activator is a silane coupling agent, which are used to create compatible interferences between non-compatible substances. Delayed surface activators create a bond between the subsequent material and the polymer resulting in a slow release of the subsequent material over time in order to provide a lasting effect. In the present embodiment, a selective silane coupling agent that is compatible with paraffin type wax is reacted under conditions with herbal oil and a wax emulsion. Other coupling agent modification treatment processes can also be contemplated.

In this embodiment, the delayed surface activator migrates to the surface of the inside surface of the glove using wax particles. In step 310, a delayed surface activator, such as a silane coupling agent, and a surface migration agent, such as herbal oil and a wax emulsion, is reacted. The surface migration agent can also be referred to as the surface adhesion promoter. The silane coupling agent is treated with de-ionized water at about 50 centigrade for about 30 minutes. The silane coupling agent and de-ionized water solution is stirred in a water bath at a range of 60 to 100 rpm. Then, paraffin wax emulsion in water is added to the hydrolyzed silane coupling agent. This mixture is heated to about 60 centigrade and cooled down to about room temperature under continuous mixing. A well-mixed solution mixture is added to the latex at least one hour prior to dipping.

In a process of incorporating olive oil extract onto a glove surface with amino-alkyl alkoxy silane derivative, dropwise addition of wax and oil emulsion into a silane coupling agent is preferred. This coupling agent process can vary depending on the subsequent treatment material, reactivity, and hydrolysis rate. The wax particles have the ability to migrate to the surface with the delayed surface activators attached to them, i.e. as the bonded mixture. A delayed surface activator can be selected to comprise an affinity to the material that is used as a subsequent treatment. The surface migration agent can also be a water based dispersion. The surface migration agent can be selected to have partial affinity to the delayed surface activator and relatively less solubility in the base polymeric material. A selected delayed surface activator can have a better affinity to the material which can be used as the subsequent treatment.

In step 312, a reacted delayed surface activator and a surface migration agent can be continuously stirred for about 30-60 minutes, more preferably 40-50 minutes to allow sufficient hydrolysis. The reaction mixture can be kept under continuous mixing for about 2 hours to allow complete hydrolysis of the delayed surface activator. Mild warming at about 45-60 centigrade of the system can expedite the hydrolysis reaction. There is a pre-mature polymeric compound comprising stabilizers, curing agents, activators, accelerators and color pigments. For example, the pre-mature polymeric compound comprises a non-ionic surfactant at about 0.2-0.5 parts, KOH at about 0.1-0.6 parts as stabilizers, about 0.5-2.0 sulfur, 0.05-0.6 parts of dithiocarbomates, 0.5-3.0 zinc oxide and color pigments. In step 314, it is preferable that the prepared hydrolyzed solution of step 312 and pre-mature polymeric compound reach room temperature before mixing. The prepared hydrolyzed solution of step 312 is slowly mixed with the pre-mature polymeric compound.

In step 316, the glove is prepared by a dipping process. The dipping process comprises a properly cleaned ceramic glove former. The glove former can be cleaned using former cleaner and water. The cleaned glove formers are dried using hot air ovens. Once dried, the glove formers are dipped into cationic coagulant solution. When the cationic coagulant solution is dried on the glove former, the glove former is dipped in the latex mixture of step 314. The cationic coagulant can be a 4%-30% water or solvent based solution of calcium chloride or calcium nitrate with 0.1-0.5 wetting agent or anti-tack agent or parting agent can be used. The compound with the delayed surface activator and migration agent is coated on top of the properly dried base layer. Due to the incompatibility of the wax particles and its comparatively poor solubility, the wax particles migrate onto the surface of the glove with a portion of the reacted silane system. The silane system is the mixture of the silane coupling agent and de-ionized water solution and wax emulsion in water. Once the wax particles migrate to the surface, the silane system reassembles and creates crosslinks under room temperature and atmospheric conditions. The migration is time dependent and can take place during or after curing, preferably for a 24-48 hours period. In step 318, the dipped glove is leached in warm water before oven curing. The leaching involves dipping the gloves in warm water that is about 55-64 centigrade for a couple of minutes to remove water soluble impurities and additional surfactants. In step 320, the glove is oven cured, which can be 30-80 minutes based on the oven and thickness of the glove. The curing temperature is about 100-150 centigrade. In step 322, the cured glove is further modified with easy donning post treatment before stripping out from the former. The post treatment 322 can be an application of a polymer, powder, or solution as desired, for example, oil and waxes, natural herbal extracts, therapeutic agents, soothing agent, a diluted acid wash, chlorination, polymer coating, cleansing agent, synthetic or natural flock and antiperspirant materials. In step 324, the resting step, stripped gloves are kept for about 24-96 hours, more preferably about 60-72 hours, resting at about room temperature. After the resting period, the affinity for both polar or non-polar subsequent material of the inside surface increases as well as the effective lifetime of the subsequent material. The desired subsequent treatment is fully or partially bounded with the core polymer.

The migration is time dependent and gloves need to rest for at least 48 hours before applying the subsequent treatment on the inside surface or outside surface as desired. After resting, step 314, the affinity for both polar and non-polar materials of the surface increases as well as the effective lifetime of the subsequent treatment once it is applied. The desired subsequent treatment is now fully or partially bonded with the core polymer making it more effective for a longer period of time.

Silane coupling agents are used to create a compatible interface between non-compatible substances. A selective silane coupling agent which is compatible with paraffin, such as wax, can be reacted under desired conditions with both herbal oil and wax emulsion. In a different embodiment, the silane coupling agent modification treatment process can be different based on material, reactivity, and hydrolysis rate. For example, in a process of incorporating olive oil extract onto an inside of a glove surface with amino-alkyl alkoxy silane derivative, dropwise addition of wax and oil emulsion into a silane coupling agent is preferred.

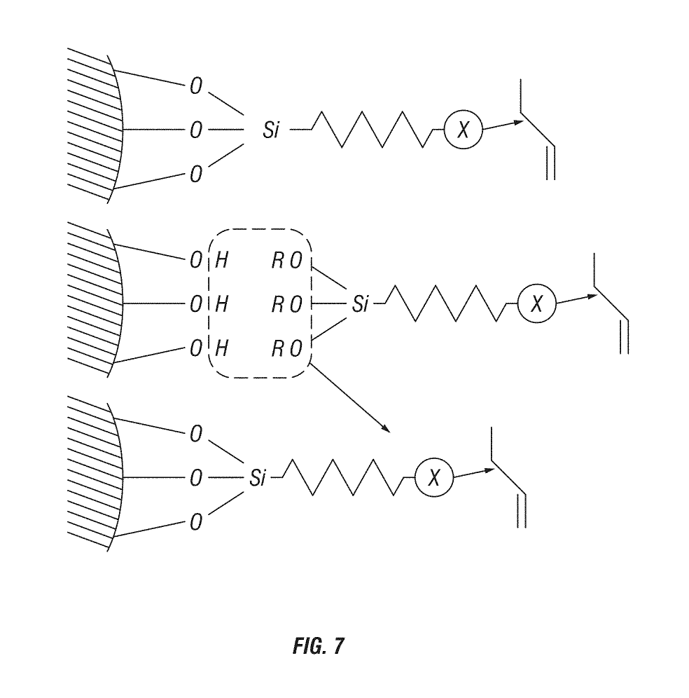

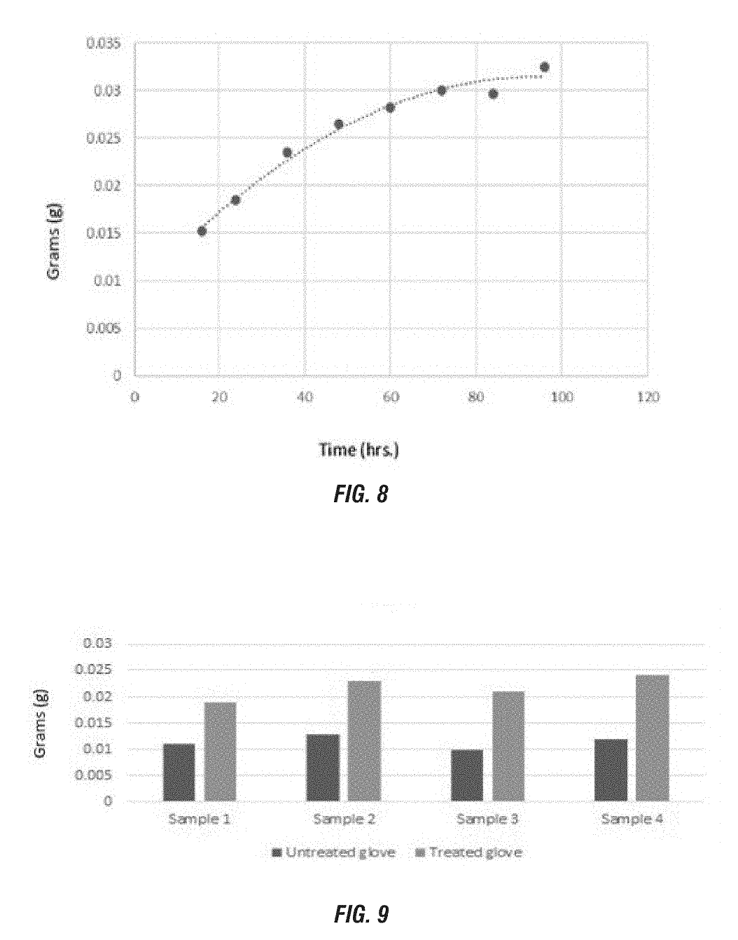

In yet another embodiment, the process 300 illustrated in FIG. 6 can also be performed on sweat control gloves. In this embodiment, the sweat control gloves are coated with an aluminum based antiperspirant in excess. A 10%-15% alcohol solution is prepared by mixing alcohol and water. The antiperspirant is dissolved in the alcohol mixture and then sprayed onto the inside surface of the glove using a mechanical sprayer. The sprayer is adjusted to coat about 1 gram-1.5 grams per glove. The treated gloves are dried in a tumbler oven to remove additional moisture. After coating with aluminum based antiperspirant, a mild washing was given to each glove. FIG. 7 illustrates a silane bond between a rubber matrix and delayed surface activator material. Results demonstrate the effectiveness of the process. The results as illustrated in FIG. 8 illustrate that at least 72 hours resting is preferred for this process. The graph shows the effective loading of subsequent material (in grams) to the inside surface of the glove against resting time by following the process illustrated in FIG. 6.

FIG. 9 is a graph illustrating the effectiveness of a delayed surface activator, according to an embodiment of the present invention. A sweat reduction material was applied to a glove surface prepared using synthetic nano clay material and a blend of bis[3-(triethoxysilyl)propyl] disulphide and 3-mercaptopropyltrimethyloxysilane hydrolyzed solution. Sweat reduction treatment was given after different resting periods. Effectiveness of the sweat reduction treatment was measured on elemental analysis method using X-ray fluorescence analyzer. The trial illustrated in FIG. 9 was conducted to identify the effectiveness of glove samples with the delayed surface activator (treated) against control samples (untreated) that were made without the delayed surface activator. The treated samples were prepared by incorporating synthetic nano clay material and a blend of bis[3-(triethoxysilyl)propyl] disulphide and 3 mercaptopropyltrimethyloxysilane system. All the samples were kept 24 hours for resting. Those rested samples were treated with a sweat reduction material in aqueous solution by dipping. Finished glove samples were tested for effective loading of quantity of sweat reduction material by elemental analysis. According to the results, glove samples with delayed surface activator shows better absorption of sweat reduction material.

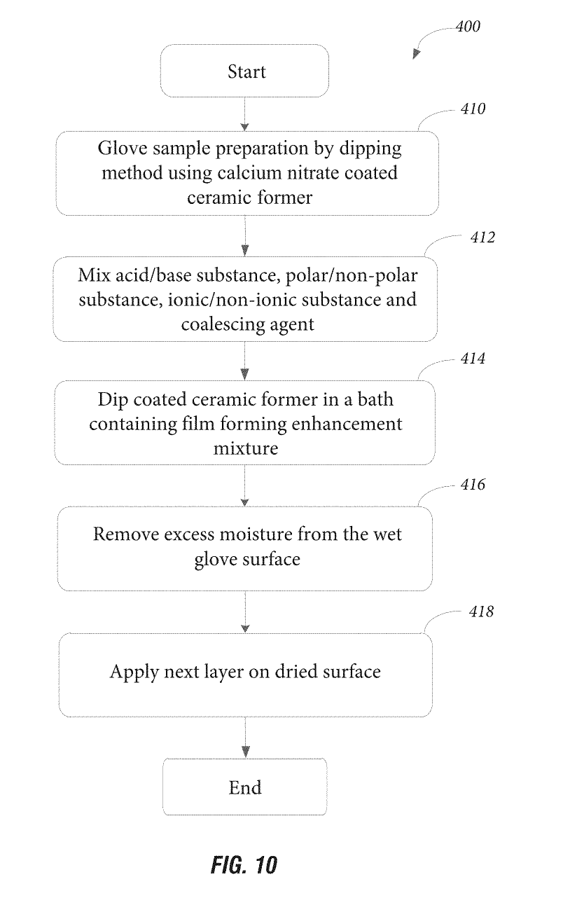

Referring now to FIG. 10, which illustrates a process 400 for surface alteration of composite, according to an embodiment of the present invention. This is yet another embodiment of minimizing the incompatibility of the inside surface with a subsequent treatment. Common acids and solvents do not swell some polymers. However, precise combinations of acids and bases and solvents can be used to alter the polymeric chains. The degree of polymer swelling is dependent on the ingredients of the surface treating composite. Size of the waves created by swelling on the surface is determined by the individual concentration of each material in the composite. The relevant composite is selected to create micro level waves on the inside surface of the glove. The selected coalescing agent is evenly spread over the micro-swollen glove surface. There is a higher tendency for partial absorption of the coalescing agent on top of the swollen surface of the glove during wave formation. The trapped and evenly spread coalescing agent aids in the secondary film formation on top of the micro swollen inside glove surface.

In step 410, the glove is prepared by a dipping method using calcium nitrate coated ceramic former. In step 412 the surface rearranging mixture, also referred to as the intermediate surface conditioning mixture or film forming enhancement mixture, is made, which comprises a mixture of a composite blend of at least one coalescing agent, polar/non-polar substance, ionic/non-ionic substance and an acid/base substance. This surface rearranging mixture enhances subsequent film forming efficacy. An example of a composite blend is 1.5 parts of acetic acid, 35 parts of methyl ethyl ketone, 40 parts of toluene, 10 parts of turpentine, 1 part of 2,4-trimethyl-1,3-pentanediol-monoisobutyrate and 12.5 parts of dichloro methane and a coalescing agent. In step 414, the coated ceramic former is dipped in a bath containing the film forming enhancement mixture from step 412 under continuous mixing at about 60-100 rpm, for about 10-12 minutes at room temperature. The dwell time of the surface rearranging composite can be 0-10 seconds, preferably 4-6 seconds. In step 416, excess solvents and water from the wet glove surface is removed by air drying. A well ventilated space is recommended for drying. In step 418, the next layer on a dried surface can be used for a desired subsequent treatment of subsequent material such as an application of a polymer, powder, or solution as desired. For example, the subsequent material may include oil and waxes, natural herbal extracts, therapeutic agents, soothing agent, a diluted acid wash, chlorination, polymer coating, cleansing agent, synthetic or natural flock and antiperspirant materials.

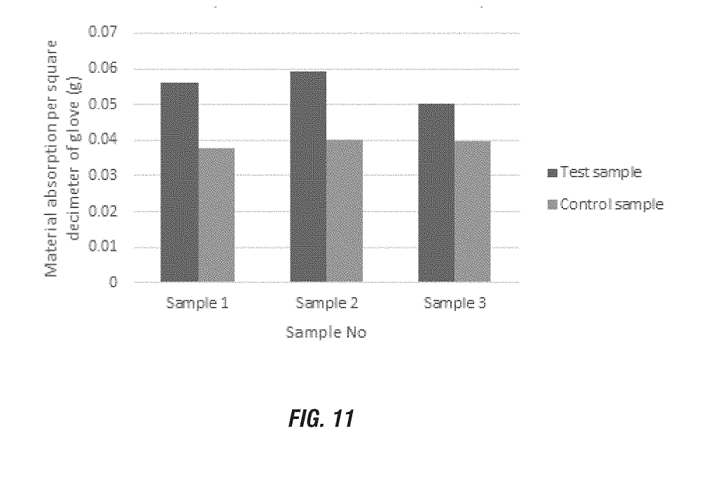

The graph illustrated in FIG. 11 compared the efficiency of material absorbance using a surface altering chemical composite, according to an embodiment of the present invention. An experimental confirmation for the effective addition of material on a swollen surface using the chemical composite was performed. In the test sample, a temporary surface opening (swelling) and material incorporation process is taken place compared to the control, which remains unchanged. The purpose of this experiment is to show the role of a surface altering agent, dimethyl sulfoxide (DMSO), in this embodiment. In this experiment, a cooling agent was incorporated to the inside surface of the glove to provide a cooling sensation to the end user. The cooling substance was dissolved in a 2% acetic acid solution for the control sample. The test sample was prepared with the same cooling material, in 1% aqueous DMSO and 2% acetic acid solution in water. (see Table 3). The surface altering agent, DMSO, in the test sample swells the exposed (inner) surface of the glove and the cooling agent is trapped in the swollen area. Acetic acid provides a low and suitable pH to shrink the swollen layer. The treated sample and the control samples were dried to evaporate excess liquids and checked for material gain. According to the test results there was a 41.9% increase in weight gain in the test sample. (see Table 4).

TABLE 3

| | |
|---|---|
| Formulation of | 1% surface altering agent + |
| the test sample | 4% cooling agent + 2% acetic acid |
| Formulation of the | 4% cooling agent + 2% acetic acid |
| control sample | |

TABLE 4

| Average weight gain | Average weight gain | Weight gain |
|---|---|---|
| in test sample | in control sample | difference (%) |
| 0.0553 | 0.0391 | 41.9% |

The surface rearranging mixture can perform at least one function out of the following: remove any dirt materials, swell and fix the surface, facilitate better deposition of coalescing agent, or expand the effective surface area of the inside surface. The composite mixture facilitates the effective application of a subsequent treatment by altering the surface characteristics across the interface by developing more favorable conditions for subsequent coatings.

While particular embodiments of the present invention have been shown and described, it will be obvious to those of skills in the art that based upon the teachings herein, changes and modifications may be made without departing from this exemplary embodiment(s) of the present invention and its broader aspects. Therefore, the appended claims are intended to encompass within their scope all such changes and modifications as are within the true spirit and scope of this exemplary embodiment(s) of the present invention.

Throughout the description and drawings, example embodiments of the present invention are given with reference to specific configurations. It will be appreciated by those of ordinary skill in the art that the present invention can be embodied in other specific forms. Those of ordinary skill in the art would be able to practice such other embodiments of the present invention without undue experimentation. The scope of the present invention, for the purpose of the present patent document, is not limited merely to the specific example embodiments of the present invention or alternatives of the foregoing description.

What is claimed is:

1. An elastomeric article comprising at least one outer layer and at least one inside layer, the inside layer having a composition that enhances an application of a subsequent material to a dried skin-facing surface of the inside layer for increased effectiveness of the subsequent material on the skin, the composition comprising:
a plurality of surface voids and cracks developed by at least one substance that is evaporated to create the surface voids and cracks at a temperature where polymer cross links are generated.

2. The elastomeric article of claim 1, wherein the at least one substance is a semi-reversible water absorption material.

3. The elastomeric article of claim 1, wherein the at least one substance is an aqueous dispersion of fumed silica.

4. The elastomeric article of claim 3, wherein the aqueous dispersion of fumed silica is about 2% to 10% w/w.

5. The elastomeric article of claim 4, wherein the aqueous dispersion of fumed silica is preferably 5% to 7% w/w.

6. The elastomeric article of claim 1, wherein the composition further comprises a poly acrylate salt solution.

7. The elastomeric article of claim 6, wherein the composition further comprises a methyl cellulose solution.

8. The elastomeric article of claim 7, wherein the methyl cellulose solution is about 0.5% to 1% w/w.

9. The elastomeric article of claim 3, wherein the fumed silica is selected from a group consisting of poly vinyl alcohol, hydrated poly vinyl acetate, sodium poly acrylate, carboxylated methyl cellulose, polyacrylamide, polyacrylate, polyacrylic acid, and polymethacrylate.

10. An elastomeric article comprising at least one outer layer and at least one inside layer, the inside layer having a composition that enhances an application of a subsequent material to a dried skin-facing surface of the inside layer for increased effectiveness of the subsequent material on the skin, the composition comprising:
one or more substances bonded with a delayed surface activator, hereinafter “bonded mixture”, the bonded mixture migrated to the skin-facing surface of the inside layer, and bondable with the subsequent material.

11. The elastomeric article of claim 10, wherein the one or more substances is a surface migration agent.

12. The elastomeric article of claim 10, wherein the delayed surface activator is a silane coupling agent.

13. The elastomeric article of claim 12, wherein the bonded mixture comprises a reacted silane coupling agent with de-ionized water solution.

14. The elastomeric article of claim 11, wherein the surface migration agent is an herbal oil and a wax emulsion.

15. The elastomeric article of claim 11, wherein the subsequent material comprises a coating of aluminum based antiperspirant bonded with the bonded mixture.

16. An elastomeric article comprising at least one outer layer and at least one inside layer, the inside layer having a composition that enhances an application of a subsequent film to a dried skin-facing surface of the inside layer for increased effectiveness of the subsequent film on the skin, the composition comprising:
a surface preparation mixture including a coalescing agent, the mixture generating a swollen surface upon which the coalescing agent is distributed, increasing adherability of the subsequent film to the swollen surface.

17. The elastomeric article of claim 16, wherein the surface preparation mixture is a blend of organic acids, inorganic acids, and organic solvents.

18. The elastomeric article of claim 17, wherein the organic acids are selected from a group consisting of polar and non-polar solvents.

19. The elastomeric article of claim 17, wherein a concentration of the organic acids is 0.5% to 5% w/w.

20. The elastomeric article of claim 17, wherein a concentration of the inorganic acids is 0.1% to 2% w/w.
